SEQUENCE LISTING (61 sequences)

SEQ ID NO: 1 - RNA, 10 nt, Synthetic construct
  source 1..10: mol_type = other RNA
gacaacttat

SEQ ID NO: 2 - RNA, 10 nt, Synthetic construct
  source 1..10: mol_type = other RNA
gacagtttat

SEQ ID NO: 3 - RNA, 10 nt, Synthetic construct
  source 1..10: mol_type = other RNA
gacaatttat

SEQ ID NO: 4 - RNA, 10 nt, Synthetic construct
  source 1..10: mol_type = other RNA
aacaacttat

SEQ ID NO: 5 - RNA, 10 nt, Synthetic construct
  source 1..10: mol_type = other RNA
aacagtttat

SEQ ID NO: 6 - RNA, 10 nt, Synthetic construct
  source 1..10: mol_type = other RNA
aacaatttat

SEQ ID NO: 7 - RNA, 10 nt, Synthetic construct
  source 1..10: mol_type = other RNA
gacaagttat

SEQ ID NO: 8 - RNA, 10 nt, Synthetic construct
  source 1..10: mol_type = other RNA
gacactttat

SEQ ID NO: 9 - RNA, 10 nt, Synthetic construct
  source 1..10: mol_type = other RNA
gacaattgat

SEQ ID NO: 10 - RNA, 10 nt, Synthetic construct
  source 1..10: mol_type = other RNA
gacaactttt

SEQ ID NO: 11 - RNA, 10 nt, Synthetic construct
  source 1..10: mol_type = other RNA
gacaacttct

SEQ ID NO: 12 - RNA, 10 nt, Synthetic construct
  source 1..10: mol_type = other RNA
gacaacttgt

SEQ ID NO: 13 - RNA, 10 nt, Synthetic construct
  source 1..10: mol_type = other RNA
gacagtttac

SEQ ID NO: 14 - RNA, 10 nt, Synthetic construct
  source 1..10: mol_type = other RNA
gacaatttag

SEQ ID NO: 15 - RNA, 10 nt, Synthetic construct
  source 1..10: mol_type = other RNA
ataagttgtc

SEQ ID NO: 16 - RNA, 10 nt, Synthetic construct
  source 1..10: mol_type = other RNA
ataaactgtc

SEQ ID NO: 17 - RNA, 10 nt, Synthetic construct
  source 1..10: mol_type = other RNA
ataaattgtc

SEQ ID NO: 18 - RNA, 10 nt, Synthetic construct
  source 1..10: mol_type = other RNA
ataagttgtt

SEQ ID NO: 19 - RNA, 10 nt, Synthetic construct
  source 1..10: mol_type = other RNA
ataaactgtt

SEQ ID NO: 20 - RNA, 10 nt, Synthetic construct
  source 1..10: mol_type = other RNA
ataaattgtt

SEQ ID NO: 21 - RNA, 10 nt, Synthetic construct
  source 1..10: mol_type = other RNA
ataacttgtc

SEQ ID NO: 22 - RNA, 10 nt, Synthetic construct
  source 1..10: mol_type = other RNA
ataaagtgtc

SEQ ID NO: 23 - RNA, 10 nt, Synthetic construct
  source 1..10: mol_type = other RNA
atcaattgtc

SEQ ID NO: 24 - RNA, 10 nt, Synthetic construct
  source 1..10: mol_type = other RNA
aaaagttgtc

SEQ ID NO: 25 - RNA, 10 nt, Synthetic construct
  source 1..10: mol_type = other RNA
agaagttgtc

SEQ ID NO: 26 - RNA, 10 nt, Synthetic construct
  source 1..10: mol_type = other RNA
acaagttgtc

SEQ ID NO: 27 - RNA, 10 nt, Synthetic construct
  source 1..10: mol_type = other RNA
gtaaactgtc

SEQ ID NO: 28 - RNA, 10 nt, Synthetic construct
  source 1..10: mol_type = other RNA
ctaaattgtc

SEQ ID NO: 29 - RNA, 10 nt, Synthetic construct
  source 1..10: mol_type = other RNA
tctgcgacaa

SEQ ID NO: 30 - RNA, 10 nt, Synthetic construct
  source 1..10: mol_type = other RNA
ctgcgacaat

SEQ ID NO: 31 - RNA, 10 nt, Synthetic construct
  source 1..10: mol_type = other RNA
tgcgacaatt

SEQ ID NO: 32 - RNA, 10 nt, Synthetic construct
  source 1..10: mol_type = other RNA
gcgacaattt

SEQ ID NO: 33 - RNA, 10 nt, Synthetic construct
  source 1..10: mol_type = other RNA
cgacaattta

SEQ ID NO: 34 - RNA, 10 nt, Synthetic construct
  source 1..10: mol_type = other RNA
gacaatttat

SEQ ID NO: 35 - RNA, 10 nt, Synthetic construct
  source 1..10: mol_type = other RNA
acaatttata

SEQ ID NO: 36 - RNA, 10 nt, Synthetic construct
  source 1..10: mol_type = other RNA
caatttataa

SEQ ID NO: 37 - RNA, 10 nt, Synthetic construct
  source 1..10: mol_type = other RNA
aatttataat

SEQ ID NO: 38 - RNA, 10 nt, Synthetic construct
  source 1..10: mol_type = other RNA
atttataatg

SEQ ID NO: 39 - RNA, 10 nt, Synthetic construct
  source 1..10: mol_type = other RNA
tttataatgg

SEQ ID NO: 40 - RNA, 10 nt, Synthetic construct
  source 1..10: mol_type = other RNA
tctgcgacag

SEQ ID NO: 41 - RNA, 10 nt, Synthetic construct
  source 1..10: mol_type = other RNA
ctgcgacagt

SEQ ID NO: 42 - RNA, 10 nt, Synthetic construct
  source 1..10: mol_type = other RNA
tgcgacagtt

SEQ ID NO: 43 - RNA, 10 nt, Synthetic construct
  source 1..10: mol_type = other RNA
gcgacagttt

SEQ ID NO: 44 - RNA, 10 nt, Synthetic construct
  source 1..10: mol_type = other RNA
cgacagttta

SEQ ID NO: 45 - RNA, 10 nt, Synthetic construct
  source 1..10: mol_type = other RNA
gacagtttat

SEQ ID NO: 46 - RNA, 10 nt, Synthetic construct
  source 1..10: mol_type = other RNA
acagtttata

SEQ ID NO: 47 - RNA, 10 nt, Synthetic construct
  source 1..10: mol_type = other RNA
cagtttataa

SEQ ID NO: 48 - RNA, 10 nt, Synthetic construct
  source 1..10: mol_type = other RNA
agtttataat

SEQ ID NO: 49 - RNA, 10 nt, Synthetic construct
  source 1..10: mol_type = other RNA
gtttataatg

SEQ ID NO: 50 - RNA, 10 nt, Synthetic construct
  source 1..10: mol_type = other RNA
ctgcgacaac

SEQ ID NO: 51 - RNA, 10 nt, Synthetic construct
  source 1..10: mol_type = other RNA
tgcgacaact

SEQ ID NO: 52 - RNA, 10 nt, Synthetic construct
  source 1..10: mol_type = other RNA
gcgacaactt

SEQ ID NO: 53 - RNA, 10 nt, Synthetic construct
  source 1..10: mol_type = other RNA
cgacaactta

SEQ ID NO: 54 - RNA, 10 nt, Synthetic construct
  source 1..10: mol_type = other RNA
gacaacttat

SEQ ID NO: 55 - RNA, 10 nt, Synthetic construct
  source 1..10: mol_type = other RNA
acaacttata

SEQ ID NO: 56 - RNA, 10 nt, Synthetic construct
  source 1..10: mol_type = other RNA
caacttataa

SEQ ID NO: 57 - RNA, 10 nt, Synthetic construct
  source 1..10: mol_type = other RNA
aacttataat

SEQ ID NO: 58 - RNA, 10 nt, Synthetic construct
  source 1..10: mol_type = other RNA
acttataatg

SEQ ID NO: 59 - RNA, 10 nt, Synthetic construct
  source 1..10: mol_type = other RNA
cttataatgg

SEQ ID NO: 60 - skipped

SEQ ID NO: 61 - DNA, 10 bp, Homo sapiens
  source 1..10: mol_type = other DNA
gacagcttat

(19) United States
(12) Patent Application Publication
(10) Pub. No.: US 2025/0043278 A1
(43) Pub. Date: Feb. 6, 2025

(54) METHODS FOR DETERMINING INCREASED RISK OF CANCER DEVELOPMENT AND TREATING THE SAME

(71) Applicant: RAMOT AT TEL-AVIV UNIVERSITY LTD., Tel Aviv (IL)

(72) Inventors: Tamir TULLER, Herzelia Pituach (IL); Ofek SCHNITZER, Tel-Aviv (IL); Hadas ZUR, Tel-Aviv (IL); Zohar ZAFRIR, Tel Aviv (IL)

(21) Appl. No.: 18/928,324

(22) Filed: Oct. 28, 2024

Related U.S. Application Data
(62) Division of application No. 17/398,252, filed on Aug. 10, 2021, now abandoned.
(60) Provisional application No. 63/063,591, filed on Aug. 10, 2020.

(51) Int. Cl. C12N 15/11; C12N 9/22; C12N 15/67; C12Q 1/6886

(52) U.S. Cl. CPC C12N 15/111; C12N 9/22; C12Q 1/6886; C12N 15/67; C12N 2310/20

(57) ABSTRACT

The present invention is directed to a method for treating cancer in a subject determined as having the presence of a G₆₀₀>A substitution, a C₆₀₁>T substitution, or both, in the B cell lymphoma 2 (BCL2) gene of a cell.

### CROSS-REFERENCE TO RELATED APPLICATIONS

This application is a divisional application of U.S. patent application Ser. No. 17/398,252 filed Aug. 10, 2021, which claims the benefit of priority of U.S. Provisional Patent Application No. 63/063,591, titled “METHODS FOR DETERMINING INCREASED RISK OF CANCER DEVELOPMENT AND TREATING THE SAME”, filed Aug. 10, 2020, the contents of which are incorporated herein by reference in their entirety.

### REFERENCE TO AN ELECTRONIC SEQUENCE LISTING

The contents of the electronic sequence listing (RMT-P-005-US1.xml; size: 53,400 bytes; and date of creation: Oct. 27, 2024) is herein incorporated by reference in its entirety.

### FIELD OF INVENTION

The present invention is in the field of cancer therapy and diagnostics.

### BACKGROUND OF THE INVENTION

Mutations in the DNA sequence are thought to be the major cause of growths/tumors and hence cancer. Mutations in the coding region of a gene can bring about a change in the amino-acid composition (missense/nonsense mutations), or alternately influence only the nucleotide sequence, without a direct effect on the protein (synonymous mutations). While synonymous (silent) mutations are often considered as lacking effect on the protein product, there is evidence they can have crucial effect on gene activity, and even direct effect on cancer development. Moreover, synonymous mutations can influence the regulatory signals at the RNA level and/or DNA level.

Currently there are 700 genes that have been classified by COSMIC (Catalogue of Somatic Mutations in Cancer) as related to the development of cancer via mutations, amongst them BCL2. The BCL gene family regulates apoptosis and programmed cell death. One of the central members of this family is BCL2 (B-Cell lymphoma 2). This gene functions as support for cell survival (pro-survival gene) by its function in the prevention of the release of cytochrome C from mitochondria, and thus the prevention of caspase-9 activity, which induces apoptosis. As a central gene preventing apoptosis, incorrect function of BCL2 can cause the failure of the cellular death process. Overexpression of BCL2 was found to be related to large diffuse B-Cell Lymphoma (DLBCL), the most common non-Hodgkin lymphoma. Translocation of BCL2 from chromosome 18 to 14 was found as the major reason for 20-30% of DLBCL cases. Moreover, this translocation contributes to the formation of new mutations, including synonymous ones, and it was shown that there is preferred selection for certain synonymous mutations. It is noteworthy that DLBCL can be further subdivided according to the gene expression profile. One of these sub-diseases is Germinal Center B-cell like (GCB) DLBCL.

Hence, additional mechanisms need to be taken into account when examining the over expression of BCL2, such as synonymous mutations. A method for determining that a subject is at increased risk of developing cancer, such as DLBCL, due to synonymous mutations is therefore greatly needed.

### SUMMARY OF THE INVENTION

According to one aspect, there is provided a method for determining a subject is at increased risk of developing cancer, the method comprising: determining a presence of a G₆₀₀>A substitution, a C₆₀₁>T substitution, or both, in a B cell lymphoma 2 (BCL2) gene of the subject, thereby determining the subject is at increased risk of developing cancer.

According to another aspect, there is provided method for treating cancer in a subject in need thereof, the method comprising: (a) determining whether a G₆₀₀>A substitution, a C₆₀₁>T substitution, or both, are present in a BCL2 gene of a cancer cell of the subject; and (b) administering to the subject determined as having a cancer cell comprising the G₆₀₀>A substitution, the C₆₀₁>T substitution, or both, in the BCL2 gene, a therapeutically effective amount of a BCL2 inhibitor, thereby treating cancer in a subject.

According to another aspect, there is provided method for classifying or prognosing cancer in a subject in need thereof, the method comprising: determining the presence of a G₆₀₀>A substitution, a C₆₀₁>T substitution, or both, in a BCL2 gene of a cell of the cancer, thereby classifying or prognosing cancer in the subject.

In some embodiments, the presence of the G₆₀₀>A substitution, the C₆₀₁>T substitution, or both, in the BCL2 gene of the subject is indicative of the subject being at increased risk of developing cancer, compared to a control subject.

In some embodiments, increased is by at least 5% compared to a control subject.

In some embodiments, the BCL2 inhibitor is reducing the expression of the BCL2 protein, transcription of the BCL2 gene, stability of the BCL2 mRNA, activity of the BCL2 protein, or any combination thereof.

In some embodiments, the BCL2 inhibitor enables the binding of a repressor to the BCL2 gene in the subject.

In some embodiments, the BCL2 inhibitor comprises the clustered regularly interspaced short palindromic repeats (CRISPR) and CRISPR-associated protein 9 system (CRISPR-Cas9).

In some embodiments, the CRISPR-Cas9 system is capable of modifying the BCL2 gene sequence, thereby enabling binding of the repressor to the BCL2 gene.

In some embodiments, the CRISPR-Cas9 system capable of modifying the BCL2 gene sequence comprises a DNA donor, wherein the DNA donor is a polynucleotide comprising G₆₀₀, C₆₀₁, or both, and is complementary to the BCL2 gene.

In some embodiments, the repressor is musculin (MSC).

In some embodiments, the cancer comprises cells expressing MSC.

In some embodiments, the cells expressing MSC are selected from the group consisting of: B cells, cardiomyocytes, and smooth muscle cells.

In some embodiments, cancer is non-Hodgkin lymphoma.

In some embodiments, non-Hodgkin lymphoma comprises diffuse large B-Cell lymphoma (DLBCL).

In some embodiments, DLBCL comprises germinal center B-cell-like (GCB) lymphoma.

In some embodiments, the presence of the G₆₀₀>A substitution, the C₆₀₁>T substitution, or both, in the BCL2 gene of the cancer cell, is indicative of the subject being afflicted with cancer classified as having cells over-expressing BCL2.

In some embodiments, the cell of a cancer is a cell comprising expression of MSC.

In some embodiments, the cell expressing MSC is selected from the group consisting of: B cells, cardiomyocytes, and smooth muscle cells.

In some embodiments, the presence of the G₆₀₀>A substitution, the C₆₀₁>T substitution, or both, in the BCL2 gene of a cell of the cancer of the subject, is indicative of the subject having poor cancer prognosis.

In some embodiments, the herein disclosed method further comprises providing a sample from the subject and performing the determining in the sample.

In some embodiments, the sample comprises DNA of the subject.

In some embodiments, the DNA of the subject comprises DNA of the cancer.

In some embodiments, the sample is devoid of RNA in sufficient amounts, quality, or both, for expression analysis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes vertical bar graphs showing that the mutations are distributed relatively uniformly across the different lengths defined by the start codon (position 0,upper graph). When focusing on the first 100 nucleotides (lower graph), it can be observed that though the mutations are distributed uniformly, the graph is noisier when the observation is made closer to the start codon, (the graph is normalized according to the number of theoretically possible mutations in those distances from the start codon).

FIG. 2 includes vertical bar graphs showing that among repetitive mutations, the rate of mutations is higher closer to the start codon (upper graph). In the lower graph, which focuses on the first 100 nucleotides after the start codon, three peaks that are not adjacent to the start codon (at distances 21-23 and 63-68) are observable. From an in-depth examination of synonymous mutations at these distances, it arises that most of them (83%) are mutations in the BCL2 gene, that occurred in germinal center B cell lymphoma (GCB) patients, a sub-type of large diffuse B-Cell Lymphoma (DLBCL).

FIG. 3 includes a histogram showing that out of only 4 cases where the synonymous mutations repeated in more than 8 patients, 3 are in the gene BCL2, thus this is indeed rare.

FIG. 4 includes graphs showing that the BCL2 gene is found on the complementary strand and contains at its center a large intron (the upper axis depicts the gene with no mutations, where the triangle and rectangle depict the start and stop codon in the gene, respectively). In the second axis the gene with mutations dispersed across it is presented, and mutations 600 and 601 can be found in the first exon (on the right). It appears there is a large concentration of mutations on this exon, and when examining the exon coding region (fourth axis in the graph) it is shown that mutations 600 and 601 are the mutations with the highest number of repeats (the number of mutations in an area is portrayed by the circle side).

FIG. 5 includes a histogram of a group of 8 GCB patients randomly sampled 10,000 times, wherein the average expression level of BCL2 for each group was measured. All the groups' results are shown. The line depicts the average BCL2 expression level in the group that contains all 8 patients with one of the suspect mutations. It is clear that among patients that have one of the suspect mutations there BCL2 is significantly overexpressed (p=0.016).

FIG. 6 includes a non-limiting diagram depicting an operation order for musculin (MSC) identification.

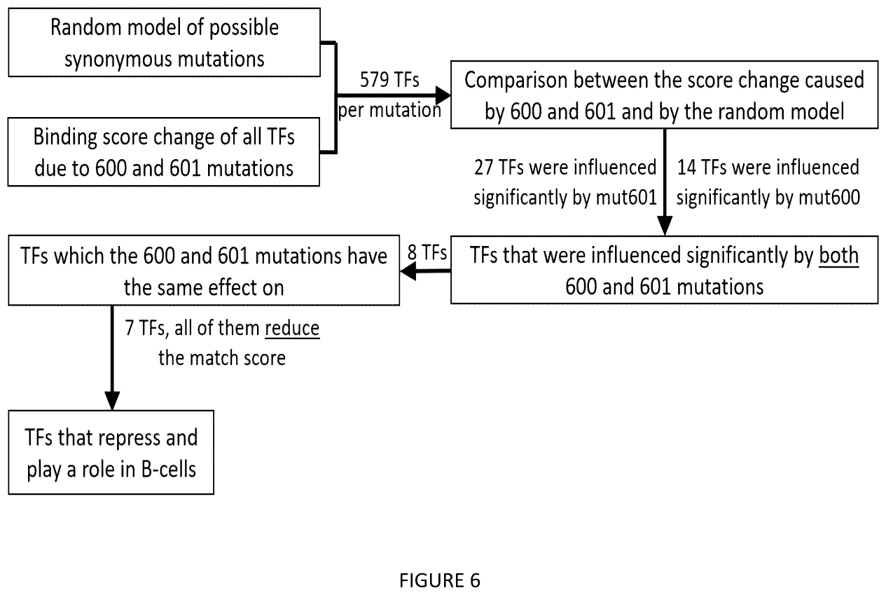

FIG. 7 includes a histogram showing the effect of mutation 600 on the possible binding results of all the transcription factors (the PSSM of transcription factors is based on JASPAR). The line depicts the effect on MSC (p=3.5·10⁻³).

FIG. 8 includes a histogram showing the effect of each of the random mutations from the random model on its binding to MSC in its region. The line depicts the effect of mutation 600 (p<8.3·10⁻³).

FIG. 9 includes a histogram showing the effect of the 601 mutation on each of the transcription factors. The line depicts the effect on MSC (p=3.5·10⁻³).

FIG. 10 includes a histogram showing the comparison of the effect of the 601 mutation on MSC binding (as compared to all the mutations in a random model). The line depicts the effect of mutation 601 (p<0.011).

FIG. 11 includes a graph of a sequence LOGO (SEQ ID NO: 60) representing the binding site necessary for MSC, where below the graph the original sequence in this region is shown (SEQ ID NO: 61), and mutations 600 and 601 are further indicated below the original sequence. Prior to the mutations, the sequence in this region matches the LOGO well, with emphasis on full compatibility at the center of the region. However, mutations 600 and 601 change the sequence to completely incompatible letters (appear with probability 0 in the PSSM).

FIG. 12 includes a graph showing a mapping of all possible windows (whose score is equal or better than the suspected site) across the gene. The size of the triangle signifies how compatible the site is to the PSSM, and its orientation indicates which strand. The play and the stop (white triangle on its side, and white rectangle), depict the start and stop codon in the gene.

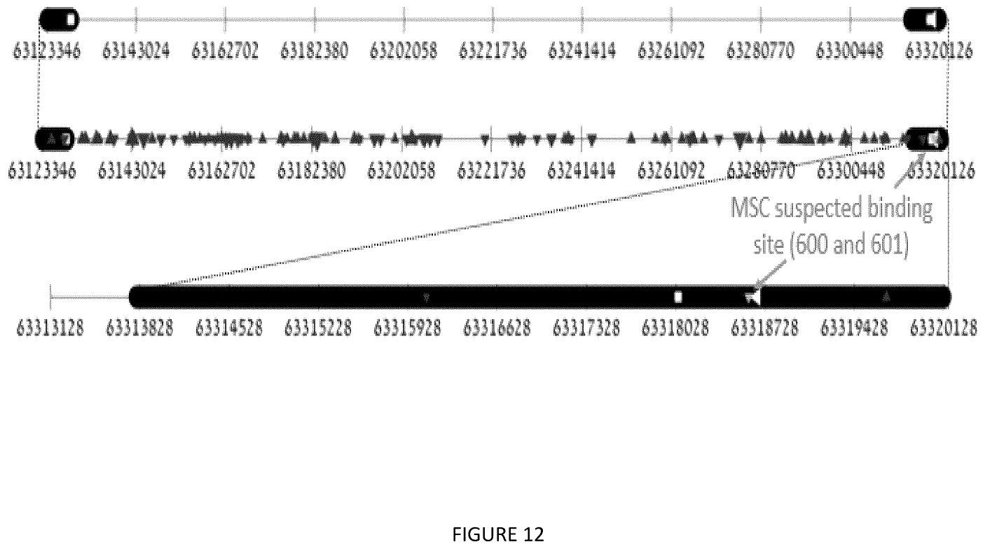

### DETAILED DESCRIPTION OF THE INVENTION

By a first aspect, there is provided a method for determining a subject is at increased risk of developing cancer, the method comprising: determining the presence of G₆₀₀>A substitution, C₆₀₁>T substitution, or both, in the B cell lymphoma 2 (BCL2) gene of the subject, thereby determining the subject is at increased risk of developing cancer.

As used herein, “G₆₀₀>A substitution” refers to a substitution of a guanine with an adenine, wherein the guanine is located on chromosome 18 at position 63318600.

As used herein, “C₆₀₁>T substitution” refers to a substitution of a cytosine with a thymine, wherein the thymine is located on chromosome 18 at position 63318601.

As used herein, the G₆₀₀>A substitution and the C₆₀₁>T substitution are within the BCL2 gene.

In some embodiments, the BCL2 gene is the BCL2 gene of a mammal. In some embodiments, the mammal is a primate. In some embodiments, the primate is human.

In some embodiments, the human BCL2 gene comprises or consists of a polynucleotide sequence, as disclosed in accession number NM_000657.2.

In some embodiments, the method further comprises providing a sample from the subject and performing the determining of the presence of the aforementioned substitutions in the sample.

According to some embodiments, there is provided a method for detecting BCL2 gene overexpression in a subject, the method comprising: providing a sample comprising a cell of the subject; and detecting whether BCL2 gene is overexpressed in the cell by determining the presence of a G₆₀₀>A substitution, a C₆₀₁>T substitution, or both in the BCL2 gene in the cell.

In some embodiments, the sample is derived or obtained from the subject.

In some embodiments, the sample comprises a cell, a tissue, or both, derived or obtained from the subject. In some embodiments, the sample comprises DNA extracted from a cell or tissue derived or obtained from the subject. In some embodiments, the sample comprises DNA of the subject. In some embodiments, the sample comprises DNA of the cancer.

In some embodiments, the sample comprises low levels of RNA, wherein the low levels are insufficient for expression analysis. In some embodiments, the sample comprises low quality RNA, wherein the low quality is insufficient for expression analysis. In some embodiments, the sample comprises at least 90%, at least 95%, or at least 99% degraded RNA, or any value and range therebetween. In some embodiments, the sample is devoid of RNA.

Methods for determining the presence of a mutation or substitution in the DNA are common and would be apparent to one of ordinary skill in the art. Non-limiting examples for determination methods include, but are not limited to, PCR, Southern-blot, fluorescent in-situ hybridization, dot-blot, sequencing (for example, Sanger), next generation sequencing, and others.

In some embodiments, determining the presence of the aforementioned mutations or substitution does not comprise or does not require reverse transcription.

In some embodiments, determining is determining in-vitro or ex vivo. It would be apparent to a person of ordinary skill in the art that determining in-vitro or ex vivo is performed in a tube, a plate, or any equivalent thereof, and not in a subject's body.

In some embodiments, the presence of the G₆₀₀>A substitution, the C₆₀₁>T substitution, or both, in the BCL2 gene of the subject is indicative of the subject being at increased risk of developing cancer, compared to a control subject.

By another aspect, there is provided a method for determining a subject is at increased risk of developing cancer, the method comprising: determining the presence of C₆₄₃>A, T, or G substitution in the B cell lymphoma 2 (BCL2) gene of the subject, thereby determining the subject is at increased risk of developing cancer.

As used herein, “C₆₄₃>A, T, or G substitution” refers to a substitution of a cytosine with an adenine, thymine, or guanine, wherein the cytosine is located on chromosome 18 at position 63318643. In some embodiments, C₆₄₃ is substituted to adenine. In some embodiments, C₆₄₃ is substituted to thymine. In some embodiments, C₆₄₃ is substituted to guanine.

As used herein, the C₆₄₃>A, T, or G substitution is within the BCL2 gene.

According to some embodiments, there is provided a method for detecting BCL2 gene overexpression in a subject, the method comprising: providing a sample comprising a cell of the subject; and detecting whether BCL2 gene is overexpressed in the cell by determining the presence of a C₆₄₃>A, T, or G substitution in the BCL2 gene in the cell.

In some embodiments, the presence of the C₆₄₃>A, T, or G substitution in the BCL2 gene of the subject is indicative of the subject being at increased risk of developing cancer, compared to a control subject.

In some embodiments, a subject at increased risk of developing cancer has greater probability of developing cancer, compared to a control subject. In some embodiments, a subject at increased risk of developing cancer is more susceptible to develop cancer compared to a control subject.

The phrases “increased risk of developing cancer”, “greater probability of developing cancer”, and “more susceptible to develop cancer” are used herein interchangeably.

In some embodiments, the control subject is a healthy subject. In some embodiments, the control subject is a subject having low risk or no risk of developing cancer. In some embodiments, the control subject is a subject having a low probability of developing cancer. In some embodiments, the control subject is a subject unsusceptible to develop cancer. In some embodiments, the control subject is a subject having a wild type BCL2 gene. In some embodiments, the control subject is a subject having a genome devoid of the G₆₀₀>A, C₆₀₁>T, C₆₄₃>A, T, or G mutations, or any combination thereof.

In some embodiments, a subject having a genome comprising the G₆₀₀>A, C₆₀₁>T, C₆₄₃>A, T, or G, or any combination thereof, in the BCL2 gene is at high risk of developing cancer.

In some embodiments, a subject at high risk of developing cancer is at least 5%, at least 10%, at least 20%, at least 35%, at least 50%, at least 100%, at least 200%, at least 350%, at least 500%, at least 750%, or at least 1,000% more susceptible of developing cancer, compared to a control subject, or any value and range therebetween. Each possibility represents a separate embodiment of the invention. In some embodiments, a subject at high risk of developing cancer is 5-100%, 10-200%, 20-500%, 35-850%, 50-450%, 100-550%, 200-900%, 350-875%, 500-1,150%, 750-1,200%, or 250-1,000% more susceptible of developing cancer, compared to a control subject. Each possibility represents a separate embodiment of the invention.

In some embodiments, a control subject has a probability of 1% at most, 2% at most, 3% at most, 5% at most, 6% at most, 7% at most, 8% at most, 9% at most, 10% at most of developing cancer, or any value and range therebetween. Each possibility represents a separate embodiment of the invention. In some embodiments, a control subject has a probability of 1-7%, 1-8%, 1-9%, 1-10%, 2-8%, 2-9%, 2-12%, 3-9%, 4-13%, 5-10%, 3-7%, or 5-15% of developing cancer.

By another aspect, there is provided a method for treating cancer in a subject in need thereof, the method comprising: (a) determining whether a G₆₀₀>A substitution, a C₆₀₁>T substitution, or both, are present in a BCL2 gene of a cancer cell of the subject; and (b) administering to a subject determined as having the G₆₀₀>A substitution, the C₆₀₁>T substitution, or both, in the BCL2 gene a therapeutically effective amount of a BCL2 inhibitor.

By another aspect, there is provided a method for treating cancer in a subject in need thereof, the method comprising: (a) determining whether a C₆₄₃>A, T, or G substitution is present in a BCL2 gene of a cancer cell of the subject; and (b) administering to a subject determined as having the C₆₄₃>A, T, or G substitution, in the BCL2 gene a therapeutically effective amount of a BCL2 inhibitor.

In some embodiments, the BCL2 inhibitor is reducing: expression of the BCL2 protein, transcription of the BCL2 gene, stability of the BCL2 mRNA, activity of the BCL2 protein, or any combination thereof.

In some embodiments, the BCL2 inhibitor directly or indirectly inhibits transcription of the BCL2 gene. In some embodiments, the BCL2 inhibitor binds to the BCL2 gene. In some embodiments, the BCL2 inhibitor binds to the promoter of the BCL2 gene. In some embodiments, the BCL2 inhibitor binds to a transcription enhancer of the BCL2 gene. In some embodiments, the BCL2 inhibitor binds to the BCL2 transcript or mRNA. In some embodiments, the BCL2 inhibitor binds to the BCL2 mature mRNA or pre-mRNA. In some embodiments, the BCL2 inhibitor binds to the BCL. In some embodiments, by binding to any one of the aforementioned elements, the BCL2 inhibitor enables the binding of a repressor to the BCL2 gene in a cancer cell of the subject. In some embodiments, the BCL2 inhibitor enables the binding of a repressor to the BCL2 gene in a cancer cell of the subject by modifying the sequence of the BCL2 gene. In some embodiments, the repressor does not bind to a BCL2 gene comprising the G₆₀₀>A substitution, the C₆₀₁>T substitution, or both. In some embodiments, the BCL2 inhibitor substitutes an A₆₀₀>G, a T₆₀₁>C, or both, so as to enable a repressor to bind to the BCL2 gene. In some embodiments, the BCL2 inhibitor enables the binding of a repressor to the BCL2 gene by hybridizing to the sequence of the BCL2 gene. In some embodiments, the BCL2 inhibitor is an antibody having specific affinity to the BCL2 protein. In some embodiments, a BCL2 inhibitor antibody is capable of neutralizing the activity of the BCL2 protein in a cancer cell of the subject.

In some embodiments, a BCL2 inhibitor is a polynucleotide. In some embodiments, a polynucleotide comprises DNA, RNA, LNA, or any hybrid thereof. In some embodiments, the BCL2 inhibitor is an RNA interfering (RNAi) polynucleotide. In some embodiments, the RNAi polynucleotide is selected from: short hairpin RNA (shRNA), small interfering RNA (siRNA), double stranded RNA (dsRNA), microRNA (miRNA), and an antisense RNA.

In some embodiments, a BCL2 inhibitor is an enzyme. In some embodiments, a BCL2 inhibitor that is an enzyme is a DNA binding peptide or protein. In some embodiments, a DNA binding protein comprises the clustered regularly interspaced short palindromic repeat associated protein 9 system (CRISPR/Cas9). In some embodiments, an agent according to the present invention comprises the Cas9 protein.

In some embodiments, the CRISPR-Cas9 system is capable of modifying the BCL2 gene sequence, wherein modifying is enabling the binding of the repressor to the BCL2 gene. In some embodiments, modifying comprises substituting or introducing mutations. In some embodiments, modifying comprises reverting the substituted G₆₀₀>A, C₆₀₁>T, or both, to the wildtype sequence of the BCL2 gene, i.e., A₆₀₀>G, T₆₀₁>C, or both.

In some embodiments, a CRISPR-Cas9 system capable of modifying the BCL2 gene comprises a DNA donor, wherein the DNA donor comprises a polynucleotide comprising G₆₀₀, C₆₀₁, C₆₀₀, G₆₀₁, or a combination thereof, and is complementary to the BCL2 gene. In some embodiments, a CRISPR-Cas9 system capable of modifying the BCL2 gene comprises a DNA donor, wherein the DNA donor comprises a polynucleotide comprising G₆₀₀, C₆₀₁, or both, and is complementary to the BCL2 gene.

In some embodiments, a CRISPR-Cas9 system capable of modifying the BCL2gene comprises a DNA donor, wherein the DNA donor comprises a polynucleotide comprising C₆₄₃, A₆₄₃, G₆₄₃, or T₆₄₃, and is complementary to the BCL2 gene. In some embodiments, a CRISPR-Cas9 system capable of modifying the BCL2 gene comprises a DNA donor, wherein the DNA donor comprises a polynucleotide comprising C₆₄₃ and is complementary to the BCL2 gene.

According to some embodiments, a BCL2 inhibitor that is an enzyme, such as Cas9, unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain embodiments, different protospacer-adjacent motif can be utilized. For example, the S. pyogenes system requires an NGG sequence, where N can be any nucleotide. S. thermophilus Type II systems require NGGNG (Horvath and Barrangou, 2010) and NNAGAAW (Deveau, Barrangou et al. 2008). Bioinformatic analyses have generated extensive databases of CRISPR loci in a variety of bacteria that may serve to identify additional useful PAMs and expand the set of CRISPR-targetable.

The term “single guide RNA” (sgRNA), is a 20 bp RNA molecule that can form a complex with Cas9 and serve as the DNA recognition module. sgRNA is typically designed as a synthetic fusion of the CRISPR RNA (crRNA) and the trans-activating crRNA.

In some embodiments, a sgRNA serves as a recognition module of the BCL2 gene comprising the G₆₀₀>A substitution. In some embodiments, a sgRNA serves as a recognition module of the BCL2 gene comprising the C₆₀₁>T substitution. In some embodiments, a sgRNA serves as a recognition module of the BCL2 gene comprising the G₆₀₀>A substitution and the C₆₀₁>T substitution. In some embodiments, a sgRNA serves as a recognition module of the BCL2 gene comprising the C₆₄₃>A, T, or G substitution. In some embodiments, the sgRNA initiates cis repair of the BCL2 gene, such as inducing an A₆₀₀>G substitution, a T₆₀₁>C substitution, a A₆₄₃>C substitution, a T₆₄₃>C substitution, a G₆₄₃>C substitution, or any combination thereof. In some embodiments, in a cis repair, the sgRNA is a cis-antisense or cis-sense sgRNA. In some embodiments, the sgRNA initiates trans repair of the BCL2 gene, such as inducing an A₆₀₀>G substitution, a T₆₀₁>C substitution, a A₆₄₃>C substitution, a T₆₄₃>C substitution, a G₆₄₃>C substitution, or any combination thereof. In some embodiments, in a trans repair, the sgRNA is a trans-antisense or trans-sense sgRNA.

In some embodiments, sgRNA to be utilized according to the herein disclosed method comprise the following polynucleotide sequences: GACAACTTAT (SEQ ID NO: 1); GACAGTTTAT (SEQ ID NO: 2); GACAATTTAT (SEQ ID NO: 3); AACAACTTAT (SEQ ID NO: 4); AACAGTTTAT (SEQ ID NO: 5); AACAATTTAT (SEQ ID NO: 6); GACAAGTTAT (SEQ ID NO: 7); GACACTTTAT (SEQ ID NO: 8); GACAATTGAT (SEQ ID NO: 9); GACAACTTTT (SEQ ID NO: 10); GACAACTTCT (SEQ ID NO: 11); GACAACTTGT (SEQ ID NO: 12); GACAGTTTAC (SEQ ID NO: 13); GACAATTTAG (SEQ ID NO: 14); ATAAGTTGTC (SEQ ID NO: 15); ATAAACTGTC (SEQ ID NO: 16); ATAAATTGTC (SEQ ID NO: 17); ATAAGTTGTT (SEQ ID NO: ATAAACTGTT (SEQ ID NO: 19); ATAAATTGTT (SEQ ID NO: 20); ATAACTTGTC (SEQ ID NO: 21); ATAAAGTGTC (SEQ ID NO: 22); ATCAATTGTC (SEQ ID NO: 23); AAAAGTTGTC (SEQ ID NO: 24); AGAAGTTGTC (SEQ ID NO: 25); ACAAGTTGTC (SEQ ID NO: 26); GTAAACTGTC (SEQ ID NO: 27); CTAAATTGTC (SEQ ID NO: 28); TCTGCGACAA (SEQ ID NO: 29); CTGCGACAAT (SEQ ID NO: 30); TGCGACAATT (SEQ ID NO: 31); GCGACAATTT (SEQ ID NO: 32); CGACAATTTA (SEQ ID NO: 33); GACAATTTAT (SEQ ID NO: 34); ACAATTTATA (SEQ ID NO: 35); CAATTTATAA (SEQ ID NO: 36); AATTTATAAT (SEQ ID NO: 37); ATTTATAATG (SEQ ID NO: 38); TTTATAATGG (SEQ ID NO: 39); TCTGCGACAG (SEQ ID NO: 40); CTGCGACAGT (SEQ ID NO: 41); TGCGACAGTT (SEQ ID NO: 42); GCGACAGTTT (SEQ ID NO: 43); CGACAGTTTA (SEQ ID NO: 44); GACAGTTTAT (SEQ ID NO: 45); ACAGTTTATA (SEQ ID NO: 46); CAGTTTATAA (SEQ ID NO: 47); AGTTTATAAT (SEQ ID NO: 48); GTTTATAATG (SEQ ID NO: 49); CTGCGACAAC (SEQ ID NO: 50); TGCGACAACT (SEQ ID NO: 51); GCGACAACTT (SEQ ID NO: 52); CGACAACTTA (SEQ ID NO: 53); GACAACTTAT (SEQ ID NO: 54); ACAACTTATA (SEQ ID NO: 55); CAACTTATAA (SEQ ID NO: 56); AACTTATAAT (SEQ ID NO: 57); ACTTATAATG (SEQ ID NO: 58); and CTTATAATGG (SEQ ID NO: 59).

In some embodiments, sgRNA to be utilized according to the herein disclosed method comprises a nucleic acid sequence that is reversed and complementary to any one of the aforementioned SEQ ID Nos: 1-59. Obtaining the reversed and complementary nucleic acid sequence of any given polynucleotide sequence would be apparent to one of ordinary skill in the art. A non-limiting example includes but is not limited to the use of an online server, such as “Reverse Complement—Bioinformatics.org”, “Reverse and/or complement DNA sequences”, “DNA sequence Reverse and Complement Tool Free”, and the like.

One skilled in the art will appreciate that any Cas9 known in the art may be utilized in the methods described herein. The Cas9 (e.g., SaCas9 as described below) can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publicly available tools available to help choose and/or design target sequences as well as lists of bioinformatically determined unique gRNAs for different genes in different species, including but not limited to the Target Finder, (e.g., E-CRISP), the RGEN Tools: Cas-OFFinder, the CasFinder: Flexible algorithm for identifying specific Cas9 targets in genomes and the CRISPR Optimal Target Finder.

According to some embodiments, the method of the invention utilizes a dead-Cas9 (dCas9). The term “dCas9” as used herein refers to a Cas9 nuclease-null variant that is altered or otherwise modified to inactivate the nuclease activity. Such alteration or modification includes altering one or more amino acids to inactivate the nuclease activity or the nuclease domain. Such modification includes removing the peptide sequence or peptide sequences exhibiting nuclease activity, i.e., the nuclease domain, such that the peptide sequence or peptide sequences exhibiting nuclease activity, i.e., nuclease domain, are absent from the DNA binding protein. Other modifications to inactivate nuclease activity will be readily apparent to one of skill in the art based on the present disclosure. Accordingly, a nuclease-null DNA binding protein includes peptide sequences modified to inactivate nuclease activity or removal of a peptide sequence or sequences to inactivate nuclease activity. The nuclease-null DNA binding protein retains the ability to bind to DNA even though the nuclease activity has been inactivated. Accordingly, the DNA binding protein includes the peptide sequence or sequences required for DNA binding but may lack the one or more or all of the nuclease sequences exhibiting nuclease activity. Accordingly, the DNA binding protein includes the peptide sequence or sequences required for DNA binding but may have one or more or all of the nuclease sequences exhibiting nuclease activity inactivated.

In some embodiments, complementarity of a polynucleotide, such as a sgRNA or a donor DNA to a target polynucleotide, such as a BCL2 gene comprising one of the aforementioned substitutions, or both, is at least 75%, at least 85%, at least 90%, at least 95%, at least 97%, at least 99%, or 100% complementary, or any range and value therebetween. Each possibility represents a separate embodiment of the invention. In some embodiments, complementarity of a polynucleotide, such a sgRNA to a target polynucleotide, such as the BCL2 gene comprising one of the aforementioned substitutions, or both, is 70-85%, 80-90% 92-97%, 95-99%, or 97-100%. Each possibility represents a separate embodiment of the invention.

In some embodiments, the term “repressor” encompasses any peptide or protein capable of binding to a DNA sequence, for example, a silencer, and inhibit or prevent gene transcription. In some embodiments, the repressor is a transcription factor. In one embodiment, the repressor is musculin (MSC; accession number NP_005089.2). In one embodiment, the repressor is RHOXF1. In one embodiment, the repressor is FIGLA. In one embodiment, the repressor is CRX. In one embodiment, the repressor is NR2E3. In one embodiment, the repressor is NHLH1.

In some embodiments, the repressor is selected from: RHOXF1, FIGLA, CRX, NR2E3, NHLH1, FOXD2, and SOX17. In some embodiments, the repressor is selected from: RHOXF1, FIGLA, CRX, NR2E3, and NHLH1.

By another aspect, there is provided a method for classifying or prognosing cancer in a subject in need thereof, the method comprising: determining the presence of a G₆₀₀>A substitution, a C₆₀₁>T substitution, a A₆₄₃>C substitution, a T₆₄₃>C substitution, a G₆₄₃>C substitution, or any combination thereof, in a BCL2 gene of a cell of a cancer, thereby classifying or prognosing cancer in the subject.

In some embodiments, the presence of a G₆₀₀>A substitution, a C₆₀₁>T substitution, or both, in the BCL2 gene of a cancer cell of a subject, is indicative of the subject being afflicted with cancer classified as having cells over-expressing BCL2.

In some embodiments, the presence of a A₆₄₃>C substitution, a T₆₄₃>C substitution, a G₆₄₃>C substitution, in the BCL2 gene of a cancer cell of a subject, is indicative of the subject being afflicted with cancer classified as having cells over-expressing BCL2.

In some embodiments, the presence of the G₆₀₀>A substitution, the C₆₀₁>T substitution, or both, in the BCL2 gene of a cancer cell of subject, is indicative of the subject being afflicted with cancer classified as having cells expressing MSC.

In some embodiments, the presence of a A₆₄₃>C substitution, a T₆₄₃>C substitution, a G₆₄₃>C substitution, in the BCL2 gene of a cancer cell of a subject, is indicative of the subject being afflicted with cancer classified as having cell expressing MSC.

In some embodiments, the cell of a cancer is a cell comprising expression of MSC.

In some embodiments, cancer is characterized or classified based on the type or origin of cells it comprises. In some embodiments, the cancer comprises cells expressing BCL2. In some embodiments, the cancer comprises cells overexpressing BCL2. In some embodiments, the cancer comprises cells expressing MSC. In some embodiments, the cancer comprises cells overexpressing MSC. In some embodiments, the cancer comprises cells expressing or overexpressing BCL2 and MSC. In some embodiments, the cancer comprises cells having at least 2-fold, at least 3-fold, at least 5-fold, at least 7-fold, or at least 10-fold BCL2 levels, MSC levels, or both, compared to control cells, or any value and range therebetween. Each possibility represents a separate embodiment of the invention. In some embodiments, control cells are non-cancerous cells. In some embodiments, control cells are benign cells. In some embodiments, control cells are cells having reduced or negligible MSC expression levels. In some embodiments, control cells are cancer cells having reduced or negligible MSC expression levels.

As used herein, “expression levels” comprise mRNA levels, protein levels, or both.

In some embodiments, cells expressing or overexpressing MSC are selected from: B cells, cardiomyocytes, and smooth muscle cells.

In some embodiments, the presence of the G₆₀₀>A substitution, the C₆₀₁>T substitution, or both, in the BCL2 gene of a cell of a cancer of a subject, is indicative of the subject having poor cancer prognosis.

In some embodiments, the presence of a A₆₄₃>C substitution, a T₆₄₃>C substitution, a G₆₄₃>C substitution, in the BCL2 gene of a cancer cell of a subject, is indicative of the subject having poor cancer prognosis.

In some embodiments, poor cancer prognosis is compared to a control subject, as defined hereinabove.

In one embodiment, poor prognosis is having a survival probability of 1%, 2%, 3%, 4%, 5%, 6%, 7%, 8%, 9%, 10%, 15% or 25% at most, or any value and range therebetween. Each possibility represents a separate embodiment of the invention. In one embodiment, poor prognosis is having a survival probability of 0.5-10%, 1-15%, 2-10%, 3-17%, 4-18%, 5-23%, 6-15%, 7-24%, 8-25% or 9-16%. Each possibility represents a separate embodiment of the invention.

In one embodiment, poor prognosis is having a survival probability of 1%, 2%, 3%, 4%, 5%, 6%, 7%, 8%, 9%, 10%, 15% or 25% at most, or any value and range therebetween, after 1 month, 2 months, 3 months, 4 months, 6 months, 9 months, 1 year, 18 months, 2 years, 3 years, 4 years, or 5 years at most after the onset of a disease in a subject, or after a subject has been diagnosed with a disease. Each possibility represents a separate embodiment of the invention. In one embodiment, poor prognosis is having a survival probability of 0.5-10%, 1-15%, 2-10%, 3-17%, 4-18%, 5-23%, 6-15%, 7-24%, 8-25% or 9-16%, after 1 month to 6 months, 2 months to 12 months, 6 months to 1 year, 9 months to 18 months, 1 year to 3 years, 6 months to 24 months, or 1 year to 5 years after the onset of a disease in a subject, or after a subject has been diagnosed with a disease. Each possibility represents a separate embodiment of the invention.

As used herein “cancer” or “pre-malignancy” are diseases associated with cell proliferation.

In some embodiments, the subject is at risk of developing cancer. In some embodiments, the subject is afflicted with cancer. In some embodiments, the subject is diagnosed with cancer.

In some embodiments, cancer comprises non-Hodgkin lymphoma. In some embodiments, non-Hodgkin lymphoma comprises DLBCL. In some embodiments, DLBCL comprises GCB.

Non-limiting types of cancer include carcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to Hodgkin lymphoma, non-Hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refers to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, nongerminomatous or nonseminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise,” or variations such as “comprises” or “comprising,” indicate the inclusion of any recited integer or group of integers but not the exclusion of any other integer or group of integers.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

At first, the inventors performed a large search for the detection of significant synonymous mutations among cancer patients, which led to focusing on DLBCL patients and the BCL2 gene. The search was divided into three parts (a diagram of the workflow can be found at the end of this section):
1. Identifying and mapping mutations—(a) First, the inventors downloaded from International Cancer Genome Consortium (ICGC) the whole genome (WGS) of over 3,000 cancer patients. From this information the inventors only filtered mutations in genes known to COSMIC as related to cancer, in order to avoid unnecessary noise. (b) The inventors tested which of the mutations were synonymous, i.e., do not affect the protein (amino acid) sequence, only the DNA and/or RNA. (c) The inventors checked which mutations repeated themselves numerously in different cancer patients, and in that manner detected selection in cancer in favor of these mutations. (d) The inventors searched the mutations of (b) and (c) for suspected mutations which were further studied in-depth.
2. Understanding the phenotype-after identifying the suspected mutations, the inventors examined what phenotype the mutations would have on the patient, with the following tests—(a) Test RNA expression levels (RNA-Seq), in order to understand if the mutations influence gene expression levels. (b) Examining the gene's mutations distribution, in order to verify whether there is a significant selection on these mutations, and hence the plausibility that they have influence. (c) Testing if the mutations appear in the 1,000 genomes database, which contains the genomic sequencing of random people representing the general population, thus if the mutation does not appear in it, but does among numerous cancer patients, it is suggested to be associated with the disease.
3. Analyzing the mechanism-in order to delineate the mechanism by which these mutations affect the disease. Within this framework the inventors examined the ability of the following factors to attach to DNA or RNA, before or after the synonymous mutations—(a) RNA binding proteins (RBPs)—proteins which attach to the RNA and affect its expression, thus a mutation at their binding site can cause a change in the expression levels of the gene. (b) microRNA (miR)-short RNA sequences which are used for verification of gene expression, via their attachment to the RNA sequence. (c) Transcription factors-proteins which bind to DNA sequences and affect the transcription of a certain gene-by triggering the transcription (activators) or restraining it (repressors).

In parallel to these tests, the inventors performed a thorough literature review to examine previous relations to the phenotype or the mechanism related to these mutations.

### EXAMPLE 1

### Identification of Suspected Mutations

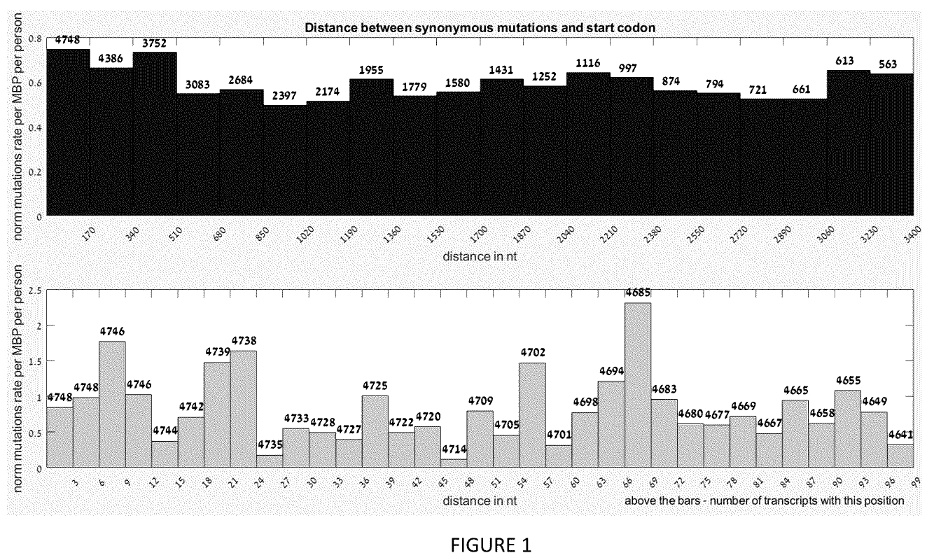

First, the inventors mapped all the synonymous mutations (for all known genes associated with cancer among all patients) according to their distance from the start codon, in order to identify anomalies or certain regularities. All mutations were found to be distributed relatively uniformly across the different lengths defined by the start codon (FIG. 1). When focusing on the first 100 nucleotides (lower graph), uniformity was lost and the landscape of mutations appeared to be somewhat noisy (FIG. 1).

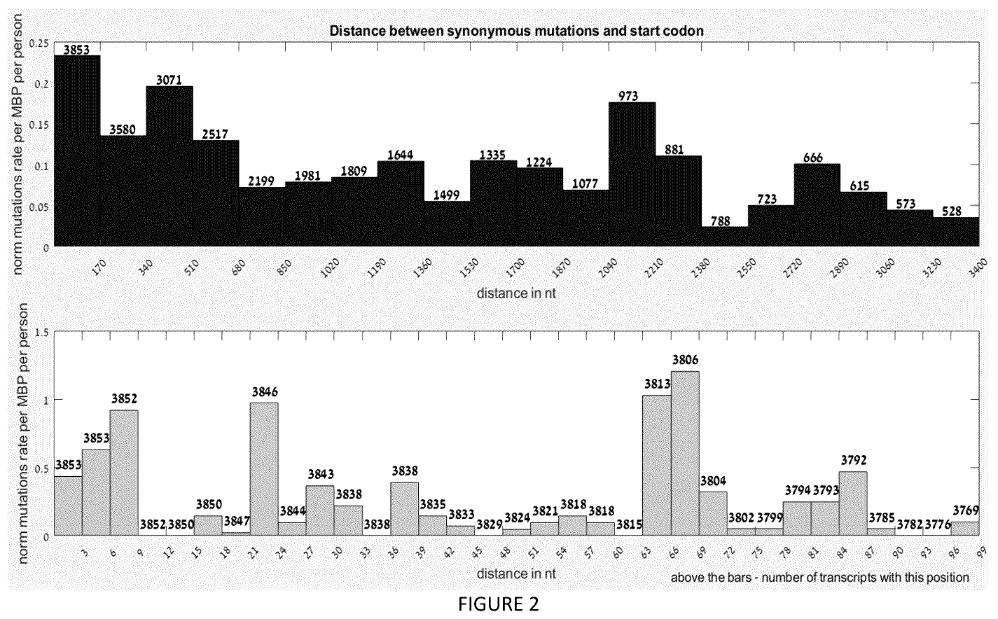

Therefore, in order to deal with the noise created, the inventors decided to filter and examine only repetitive mutations, i.e., mutations which repeated themselves in at least two patients. The rate of mutations was found to be higher when the mutation was closer to the start codon (FIG. 2). When focusing on the first 100 nucleotides after the start codon, a peculiar phenomenon was observed: three peaks that were not adjacent to the start codon (at distances 21-23 and 63-68). From an in-depth examination of synonymous mutations at these distances, it arises that most of them (83%) are mutations in the BCL2 gene that occurred in GCB patients, a sub-type of DLBCL. The mutations are: (1) 18:63318600 substitution from G to A found in 9 patients (termed mutation 600)—the cause of a peak at distance 66-68; (2) 18:63318601 substitution from C to T found in 14 patients (termed mutation 601)—the cause of a peak at distance 63-65; and (3) 18:63318643 substitution from C to A/T/G found in 10 patients-the cause of a peak at distance 21-23.

All the mutations were identified in a research regarding GCB-DLBCL, in which 241 patients had participated. The meaning is that more than 10% of the patients had one of the discussed synonymous mutations. Thus, the inventors decided to focus on the synonymous mutations 600 and 601, which are common among GCB-DLBCL, as not only do they repeat in numerous patients, but they are adjacent and therefore possibly connected to the same mechanism. Moreover, in both mutations there was only one possible substitution among patients (at 600 from G to A, and at 601 from C to T), what potentially indicated selection towards a certain type of mutations in this region.

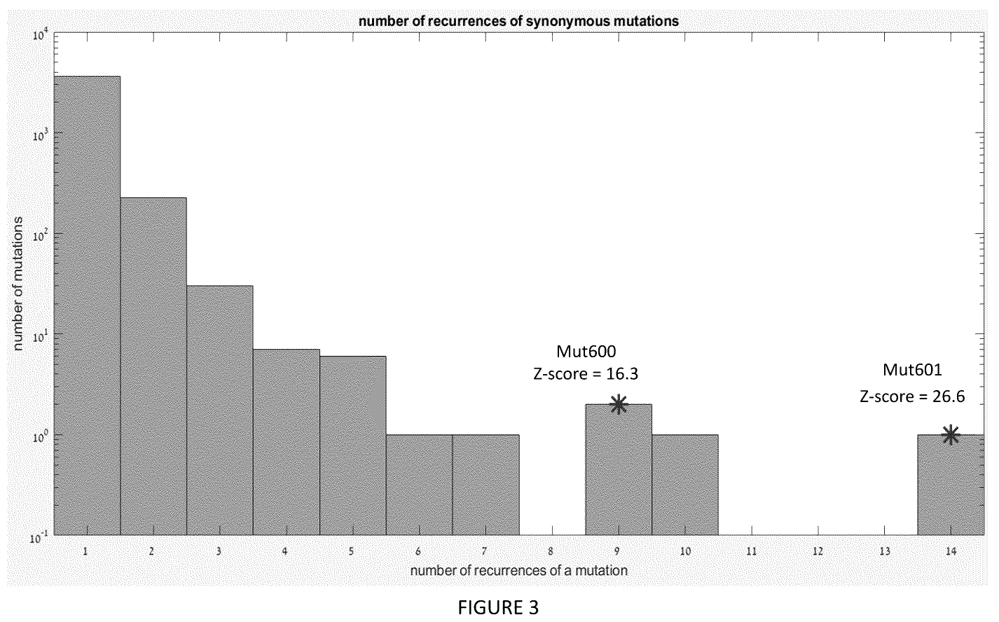

In order to validate that the repetitiveness of these synonymous mutations among this number of patients is a rare event, the inventors checked the rate of synonymous mutations which are highly repetitive (in all genes and all patients). Out of only 4 cases where the synonymous mutations repeated in more than 8 patients, 3 were in the gene BCL2, thus this is indeed rare (FIG. 3).

Finally, in order to validate there is significant selection for these mutations, the inventors searched other resources, whose results cannot be found in ICGC. Two cases regarding DLBCL patients were found, who also in their raw data over 10% of the patients had one of the 600 or 601 mutation.

### EXAMPLE 2

### Understanding the Phenotype

First, the inventors tested if the 600 and 601 mutations exist in the general population, via the 1,000 genomes DB. These mutations did not exist in 1,000 genomes, what strengthened the proposition that there is a relation between these mutations and cancer, specifically GCB-DLBCL.

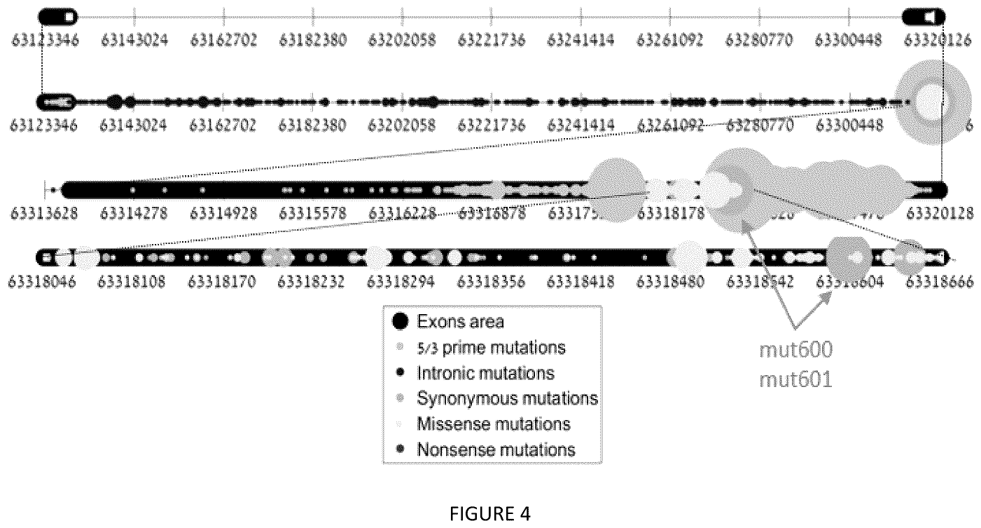

The inventors then tested the mutation dispersion and the types of mutations along the gene. The gene was found on the complementary strand and contained at its center a large intron (FIG. 4). The gene was found to comprise numerous mutations dispersed along it, and mutations 600 and 601 were found in the first exon (FIG. 4).

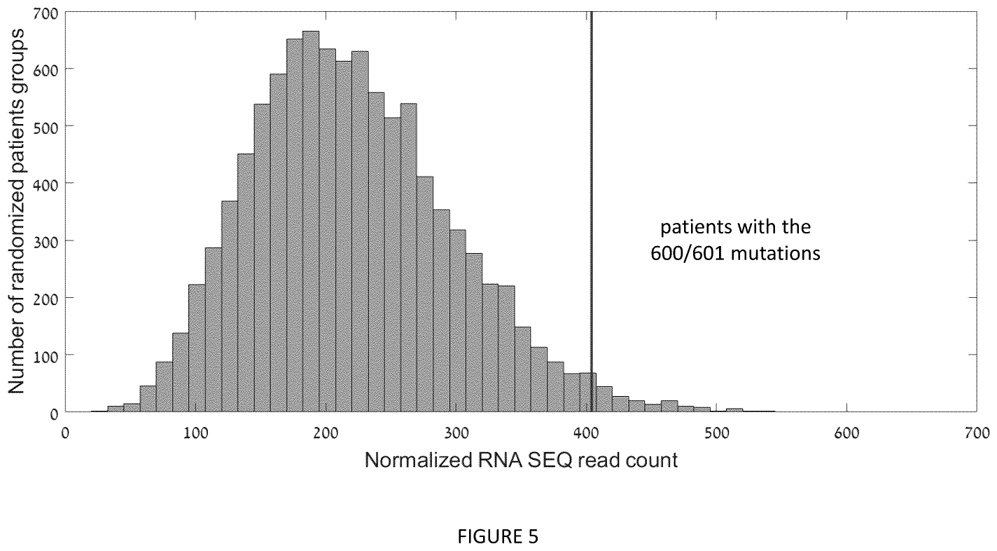

At the next stage the inventors wanted to understand how the mutations influence BCL2 and what phenotype they create. For this purpose, the inventors utilized RNA-Seq of GCB patients available in ICGC. Of notion is the fact that out of 241 GCB patients for which whole genome sequencing was available, 109 also had RNA-Seq measurements, out of which 8 had one of the suspected mutations (600 or 601). Thus, the inventors randomly sampled 10,000 times a group of 8 GCB patients and measured the average expression level of BCL2 for each group (FIG. 5). Among patients that had one of the suspect mutations there was observed a statistically significant overexpression of BCL2 (p=0.016; FIG. 5).

Therefore, it appears that unlike the general population (as portrayed in 1,000 genomes), there is selection among GCB patients for the suspect synonymous mutations (600 and 601), and these mutations cause the overexpression of BCL2 which prevents apoptosis, and in this manner aid cancer by creating cells that cannot be induced to undergo apoptosis.

### EXAMPLE 3

### Mechanism Analysis

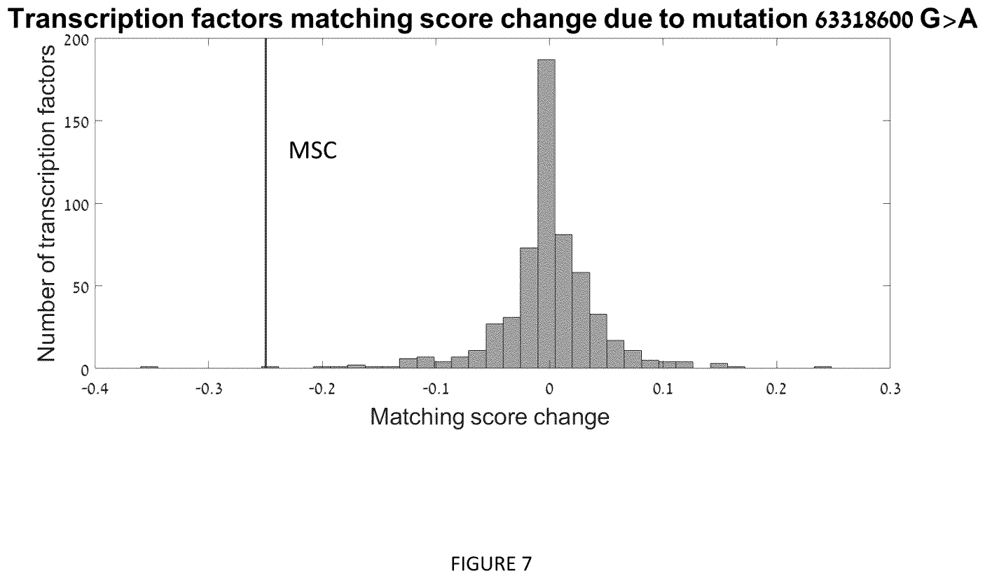

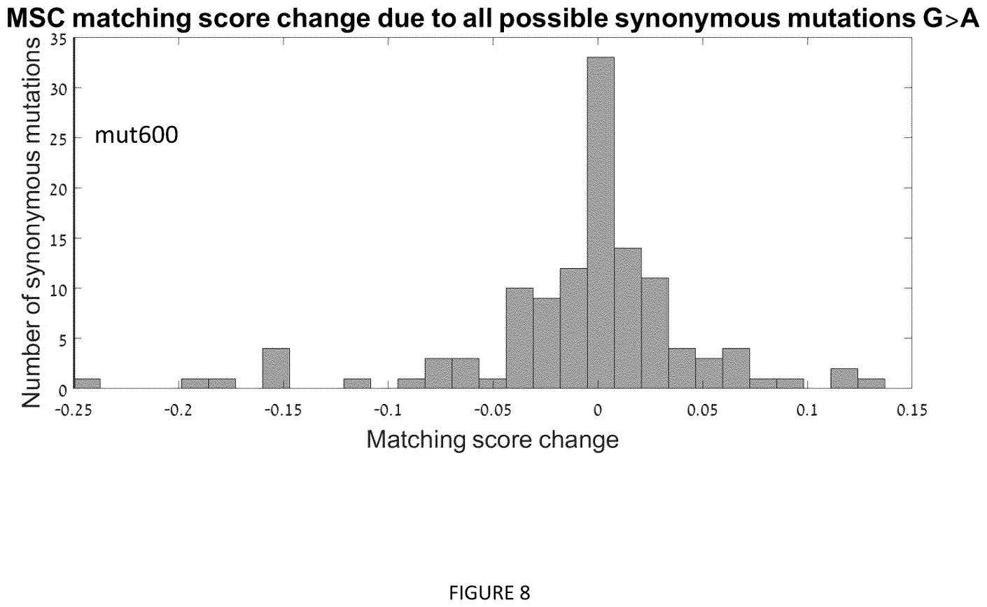

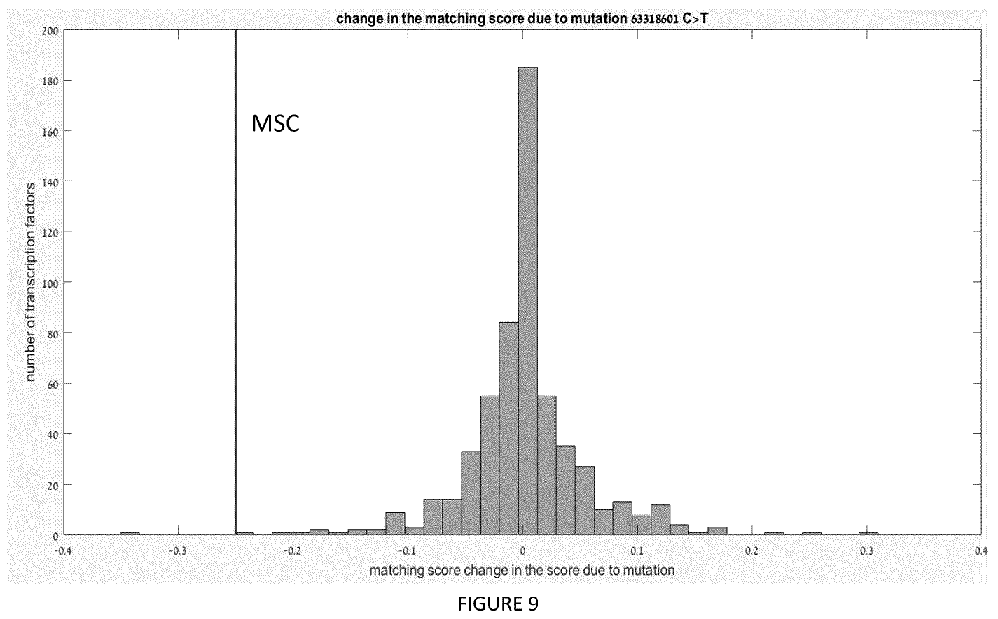

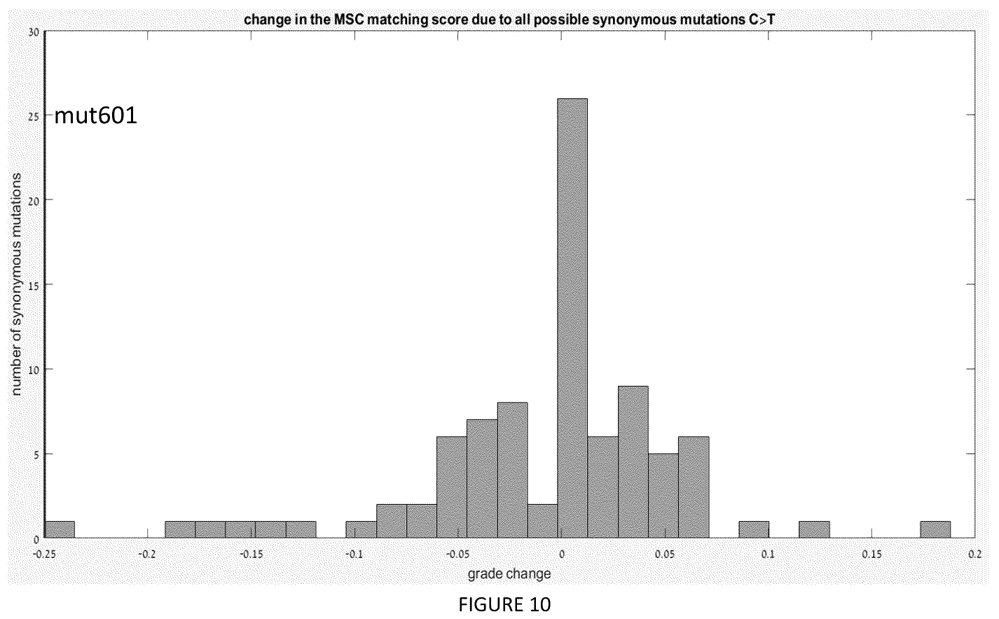

In order to find the mechanism by which these mutations affect BCL2 expression, the inventors examined three possible mechanisms: microRNA (miRNA), RNA binding protein (RBP), and transcription factors. For each of the mechanisms the inventors moved along the DNA/RNA sequence using a sliding window and tested the ability of each of the mechanisms to attach to the region prior and following each of the mutations respectively (for transcription factors the inventors tested both strands as the effect is at the DNA level). The inventors then devised a random model which was composed of identical random mutations (synonymous mutations from other regions in the gene where there is identical substitution to the one in the mutations) and compared the change that the suspected mutations created to those in the random model, in order to identify regulatory factors that the suspected mutations influenced more than the mutations in the random model. It is noteworthy that these tests were performed for each of the mutations (600 and 601) separately. The tests did not garner statistically significant results for miRNA or RBP, however, when testing transcription factors, the transcription factor musculin (MSC, ABF1; FIGS. 7-10) was significantly affected by each mutation separately, where the mutations significantly reduced its ability to attach to the DNA. From a literature review it arises that this factor constitutes a repressor in B cells of the immune system. Moreover, it was found that methylation of this gene is related to DLBCL. This is consistent with the results that indicated overexpression due to these mutations, as if it is a B cell repressor and the mutations prevent its attachment to the DNA, therefore gene overexpression is observed.

Thus, the inventors concluded that binding of the MSC repressor, which is functional/active in B cells, will be hampered by each of the respective mutations (separately) in the area, hence may induce overexpression of BCL2.

### EXAMPLE 4

### Binding Analysis

Finally, in order to validate whether the proposed mechanism (damage to the binding of the repressor MSC) is indeed the one by which the synonymous mutations affect the expression of BCL2, the inventors tested if the repressor is attached to this area prior to the mutations.

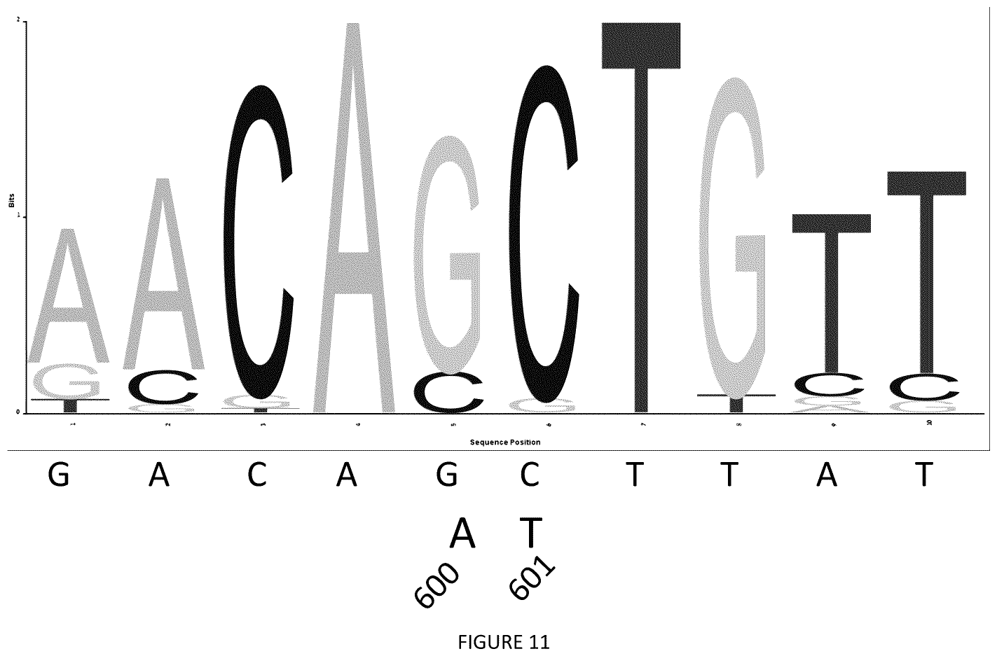

First, the inventors used LOGO which represents the binding site necessary for MSC. Prior to the mutations, the sequence in this region matched the LOGO well, with emphasis on full compatibility at the center of the region (FIG. 11). However, mutations 600 and 601 changed the sequence to completely incompatible letters (FIG. 11).

Thus, the inventors hypothesized that the adaptability of the region prior to the mutations is indeed extraordinary in its quality, and therefore, examined all the possible binding sites of the gene whose binding score according to the PSSM is equal or better than the suspected site. Out of 196,783 possible double windows (for both strands) in the gene, there were only 103 window sites whose compatibility was better than the suspected site (p=5.2·10⁻⁴). The inventors then looked at the possible window mapping in the gene and discovered that the suspected site was the first site in the regular strand, and there was no better site in this strand near the beginning of the gene. Thus, it appears this is a suitable site for MSC repressor binding, and there are no superior alternative sites near the beginning of the gene on this strand. Therefore, it appears that sequence aberrations at this particular site will prevent repressor binding, and subsequently induce gene overexpression.

The inventors used the methods mentioned above (mechanism logic and transcription factors expression data) for finding the relevant transcription factor out of the ones that their binding score was changed significantly. As can be seen in Table 1, only MSC, passed all the tests. The latter binding score was changed significantly due to the mutations (z-score of −5.5231), it is a repressor that its binding score declined (−5.74391), and it is highly expressed in lymphocytes (median TPM of 143.53, 9.6801 times the mean median TPM across all tissues). MSC also known as ABF-1, and it is a well-known factor expresses in lymphocytes. Methylation of MSC was shown to be correlated with DLBCL, as well as SNPs in the MSC gene itself quality

TABLE 1

| | | | | Median TPM | |
|---|---|---|---|---|---|
| | | Sum change | | in the | Median TPM in the |
| | Sum change | in binding | | tissue | tissue ‘Cells-EBV- |
| | in binding | score due to | | ‘Cells- | transformed |
| | score due to | 600 and 601 | | EBV- | lymphoctyes’/mean |
| Transcription | 600 and 601 | mutations z- | Transcription | transformed | median TPM in all |
| factor | mutations | score | factor type | lymphocytes’ | tissues |
| RHOXF1 | −7.93801 | −7.6267 | Unknown | 0.558 | 0.178 |
| MSC | −5.74391 | −5.5231 | Repressor | 143.53 | 9.6801 |
| FIGLA | −4.75537 | −4.5753 | Activator | 0.0066 | 0.0354 |
| CRX | −4.50247 | −4.3329 | Activator | 0.01 | 0.7279 |
| NR2E3 | −4.05709 | −3.9058 | Activator | 0.024 | 0.069 |
| NHLH1 | −3.52741 | −3.398 | Activator | 1.53 | 2.398 |
| FOXD2 | 5.367526 | 5.13 | Activator | 0.74 | 0.5365 |
| SOX17 | 3.63272 | 3.4667 | Activator | 0.033 | 0.0023 |

This table shows the results of the different tests done on the transcription factors with very high/low z-score due to significantly change in the matching score in 600 and 601 mutations area. The tests included the sum change in the binding score due to the mutations, the type of the transcription factor, the median TPM in lymphocytes, and the median TPM lymphocytes/mean median TPM in all tissues. In the center part of the table (columns 2-4) the inventors highlighted (in bold font) the transcription factors whose type (activator/repressor) matches the change in the binding score in order to match the BCL2 overexpression. In the right part (columns 5-6) the inventors highlighted (in bold font) the transcription factors that are highly expressed in lymphocytes (their expression in lymphocytes is higher than their mean median expression in all tissues).

### Summary

Synonymous cancerous mutations, in the coding region of the gene BCL2 were identified by large-scale data analysis. The inventors found that these mutations cause the overexpression of BCL2, and thus prevent apoptosis. Furthermore, the inventors found that the mechanism by which these mutations affect gene expression is by binding prevention of the repressor MSC to BCL2, that apparently used to bind to this site prior to the appearance of the mutations.

While the present invention has been particularly described, people skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A method for reducing susceptibility of a subject to develop cancer, compared to a control subject, the method comprising:
a. determining whether a G₆₀₀>A substitution, a C₆₀₁>T substitution, or both, are present in a B cell lymphoma 2 gene (BCL2) of a cancer cell of said subject; and
b. administering to said subject determined as having a cancer cell comprising said G₆₀₀>A substitution, said C₆₀₁>T substitution, or both, in said BCL2 gene, a therapeutically effective amount of a polynucleotide being a BCL2 inhibitor,
thereby reducing susceptibility of the subject to develop cancer.

2. The method of claim 1, wherein said polynucleotide being a BCL2 inhibitor is reducing the expression of the BCL2 protein, transcription of the BCL2 gene, stability of the BCL2 mRNA, or any combination thereof.

3. The method of claim 1, wherein the administration of said polynucleotide being a BCL2 inhibitor enables the binding of a repressor to said BCL2 gene in said subject.

4. The method of claim 3, wherein said repressor is musculin (MSC).

5. The method of claim 1, wherein said cancer comprises cells expressing MSC.

6. The method of claim 5, wherein said cells expressing MSC are selected from the group consisting of: B cells, cardiomyocytes, and smooth muscle cells.

7. The method of claim 1, wherein said cancer is non-Hodgkin lymphoma.

8. The method of claim 7, wherein said non-Hodgkin lymphoma comprises diffuse large B-Cell lymphoma (DLBCL).

9. The method of claim 8, wherein said DLBCL comprises germinal center B-cell-like (GCB) lymphoma.

10. The method of claim 1, wherein said subject determined as having said G₆₀₀>A substitution, said C₆₀₁>T substitution, or both, in said BCL2 gene of said cancer cell has a poor cancer prognosis.

11. The method of claim 1, wherein the determining is in a sample comprising DNA of said subject.

12. The method of claim 11, wherein said DNA of said subject comprises DNA of said cancer cell.

13. The method of claim 11, wherein said sample comprises low levels of RNA, low quality RNA, or both, that are insufficient for expression analysis, or is devoid of RNA.

14. The method of claim 1, wherein said polynucleotide being a BCL2 inhibitor is an RNAi polynucleotide.

15. The method of claim 1, wherein said control subject has a genome comprising a wild type BCL2 or being devoid of the G₆₀₀>A substitution, the C₆₀₁>T substitution, or both.
